(19) United States
(12) Patent Application Publication
(10) Pub. No.: US 2007/0187242 A1
(43) Pub. Date: Aug. 16, 2007

(54) Electro-optical modulating display devices

(75) Inventors: Mridula Nair, Penfield, NY (US); Tamara K. Jones, Rochester, NY (US); Mary C. Brick, Webster, NY (US); Todd M. Spath, Hilton, NY (US)

Correspondence Address: Paul A. Leipold;Patent Legal Staff, Eastman Kodak Company, 343 State Street, Rochester, NY, 14650-2201, US

(73) Assignee: Eastman Kodak Company

(21) Appl. No.: 11/352,587

(22) Filed: Feb. 13, 2006

(51) Int. Cl. C07K 1/26

(52) U.S. Cl. 204/450

(57) ABSTRACT

The present invention relates to electro-optical modulating display devices and, specifically, to such displays containing oil-in-oil emulsions as the imaging material. Also disclosed is a method of forming an image by movement of liquid droplets through a continuous liquid phase, the method comprising providing an array of pixel elements, each containing at least one separate reservoir of electro-optical imaging fluid comprising a colloidally stable dispersion of an oil-in-oil emulsion.

### CROSS-REFERENCE TO RELATED APPLICATIONS

The present application is related to U.S. application Ser. No. ______ (Docket No. 91862), filed on the same date hereof by Jones et al., and entitled, “OIL-IN-OIL DISPERSIONS STABILIZED BY SOLID PARTICLES AND METHODS OF MAKING THE SAME” and to U.S. application Ser. No. ______ (Docket No. 91861), filed on the same date hereof, by Nair et al., and entitled “OIL-IN-OIL EMULSIONS,” hereby incorporated by reference in their entirety.

### FIELD OF THE INVENTION

The present invention relates generally to the field of electro-optical modulating display devices and, specifically, to such displays containing oil-in-oil emulsions. In particular, the invention relates to electro-optical modulating display devices such as electrophoretic, electrowetting, and electrochromic display devices, which comprise oil-in-oil emulsions in an array of cells.

### BACKGROUND OF THE INVENTION

Electro-optical modulated display devices include display devices in which the optical state of an imaging material is modulated or changed by subjecting the imaging material to at least an electric field or the transport of electrons, for example, electrophoretic, electrowetting, and electrochromic display devices.

The electrophoretic display device, one particularly advantageous type of electro-optical modulated display, was developed as an alternative to CRT and LCD displays, particularly for portable display applications. In general, electrophoretic image displays are advantageous in that they require significantly less power than CRT displays and can be viewed over a wider field of view than LCD displays. An electrophoretic display (EPD) also offers an electronic alternative to conventional printed-paper media for many applications. Electrophoretic devices are based on the electric field induced motion of charged particles suspended in a fluid, such as charged pigment particles in an organic solvent. Unlike sheet materials containing magnetic memory areas that can be written electronically, an EPD advantageously provides a visible record for the viewer. The particles serve to either contribute a color or the absence of a color to the display.

Evans et al. (U.S. Pat. No. 3,612,758) describe the utilization of electrophoresis as a basis for a passive EPD. In one embodiment, a fluid suspension composed of a colored solvent and charged pigment particles, is enclosed between two plates. When a voltage potential is applied across a pair of electrodes, the electric potential draws the charged particles to a particular electrode. When no voltage is applied, the charged particles remain dispersed in the dispersion fluid, the color of the pixel being controlled by whether the charged particles are dispersed.

A number of problems were apparent with early generation EPDs, including: (a) particles tended to segregate into locations that were most frequently addressed, and (b) particles tended to settle according to orientation (due to gravity) creating a gradient of particle density in the display. Such segregation of particles resulted in inadequate service-life of the display. In order to maintain particle uniformity, isolated cells were prepared by introducing partition walls. See for example, Hopper, M and Novotny, V., IEEE Trans. Elect. Dev. 26(8), pp. 1148-1152, 1979. By isolating each cell, particle migration across the display caused by settling, electric field induced particle migration, or by fluid motion across the display was managed. More recently, so-called microcups, each filled with a particle dispersion has been described. See for example, U.S. Pat. No. 6,850,355 and US2003/0151029. Another method for making an EPD with isolated microfluidic structures is that utilizing an assembly of microencapsulated particle dispersion. See for example, Nakamura et al., Development of Electrophoretic Display Using Microencapsulated Suspension; and Drzaic et al., A Printable and Rollable Bistable Electronic Display, Society for Information Display Symposium Proceedings, 1131-1138, 1998.

EPDs using out-of-plane electrodes are also known to suffer from uneven particle deposition at electrodes. As a result of writing an image, particles assembled at electrodes tend to cluster and agglomerate resulting in a degradation of the desired reflective state and deterioration in performance over time. (See, for example, Mürau, P. and Singer, B., “The Understanding and Elimination of Some Suspension Instabilities in an Electrophoretic Display,” J. Appl. Phys. 49(9), pp. 4820-4829, 1978; Dalisa, A., “Electrophoretic Display Technology,” IEEE transactions on Electronic Devices, Vol. 24, 827-834, 1977). Agglomeration (particles sticking to each other) is usually irreversible. Clustering of particles is thought to be due to electrohydrodynamic flow effects, and is usually reversible and controllable by applied voltage and the frequency of the switching waveform (Mürau and Singer, cited above; Trau, M, Sankaran, S., Saville, D. A., and Aksay, I. A., “Pattern Formation in Nonaqueous Colloidal Dispersions via Electrohydrodynamic Flow, Langmuir Vol. 11, pp. 4665-4672, 1995; and Ristenpart, W. D., Aksay, I. A and Saville, D. A., “Assembly of Colloidal Aggregates by Electrohydrodynamic Flow: Kinetic Experiments and Scaling Analysis,” Phys. Rev. E Vol. 69, pp. 021405, 2004). Another problem that frequently arises is the irreversible sticking of particles at electrode surfaces. Such sticking to electrodes is clearly undesirable as it reduces the useful life of a display as well as the contrast ratio and image quality that can be achieved.

To view an EPD, a light source is needed. For example to view a reflective display in the dark, either a backlight system or a front pilot light system may be used. However, the presence of light scattering particles greatly reduces the efficiency of the backlight system. A high contrast in both dark and well-lit environments is difficult in parallel electrode EPDs. Additionally, the extra cost for the external lighting system and cumbersome hardware makes this option unattractive.

In order to overcome the lighting and contrast deficiencies of EPDs having out-of plane electrodes for pixel elements, in-plane electrode switching has been considered. For such in-plane electrode devices, collector electrodes are provided adjacent to and substantially in the same plane such that particles typically move substantially parallel rather than perpendicular to the face of the display (See, for example, Kishi, E. et al., Development of In-plane EPD,” SID 2000, pp. 24-27); and Liang et al., US Pat. Pub. 2003/0035198). In-plane devices have also been called “horizontal migration type electrophoretic display device,” (See U.S. Pat. No. 6,741,385). In a first transmissive or reflective state, particles are assembled on a transparent viewing electrode. In a second transmissive or reflective state, the particles are removed from the viewing electrode and collected on at least one collector electrode. The collector electrode need not be transparent and may be hidden by an external mask, or may be made narrowly so as to minimally affect the contrast between the dark (colored) and light (colored) state. A variation on the in-plane electrode arrangement is to provide collector electrodes close to partition walls and on the walls themselves. The efficiency of the backlight and contrast between dark and light state is improved, as light scattering particles are no longer in the optical path between a viewer and backlight. However, such in-plane devices still suffers from the inhomogeneous deposition of particles on viewing electrodes and incomplete clearing of particles from the viewing electrode due to particle sticking.

Regardless of the type of device, the predictable and reproducible transport of particles is critical to good device performance. A key factor in maintaining colloidal stability and reproducible particle transport is the stability of the particles' electrostatic character. Thus, it is well known in the art to provide particle dispersions with native charge or particles that acquire charge in the presence of charge agents (see for e.g. Croucher, M. D., Harbour, J., Hopper, M. and Hair, M. L., “Electrophoretic Display: Materials as Related to Performance,” Photographic Science and Engineering, Vol. 25, pp. 80-86, 1981; U.S. Pat. No. 4,298,448; US Pat. Pub. 2003/0035198). It is also known to provide pigment particles with adsorbed polymer or polymer coatings (See, for example, U.S. Pat. No. 4,298,448; U.S. Pat. No. 5,783,614; and Mürau and Singer, cited above). The development of new charge control agents remains an active research and development activity because existing charge control agents are only useful in specific dispersions and for specific electrode cell designs. For instance, a charge control agent may perform well in a out-of-plane pixelated EPD. but fails when tested in an in-plane pixilated EPD utilizing an electrical gate electrode.

The formation of stable dispersions, in itself, using particles is difficult. For example, it is difficult to match the specific densities of the EPD fluid and the solid particles to form a stable dispersion. In addition, the image response rate achieved by EPDs using charged particles is limited by the rate at which the particles can be drawn to and from the electrodes through the dispersion fluid. Hence, although invented about 30 years ago, attempts to successfully commercialize EPD technology has failed because of stability problems of solid-particle-based display.

EPD imaging materials can be divided into two main classes, encapsulated and non-encapsulated. Encapsulated mediums comprise numerous small capsules, each of which itself comprises an internal phase containing two or more different types of electrophoretically mobile particles suspended in a liquid suspension medium, and a capsule wall surrounding the internal phase. Typically, the capsules are themselves held within a polymeric binder to form a coherent layer positioned between two electrodes. Encapsulated media of this type are described for example in U.S. Pat. No. 6,822,782 to E Ink Corp., and are commonly used for EPDs with parallel electrode pixels. While encapsulated electrophoretic media are useful in EPDs with out-of-plane electrodes, they suffer from settling in the liquid medium, are complex to produce, and are not useful for EPDs with in-plane electrodes due differences in imaging mechanisms between the two types of displays.

The drawbacks for displays using solid particles are due to the fact that the particles are solids. Colloidal stability of such systems in an electric field is not optimum. Additionally, such displays are also not simultaneously optimized for contrast, speed and power. In order to achieve high contrast, these displays require high optical density from the dye solution. This is obtained by increasing the distance between opposing electrodes, thereby generating a higher volume. This in turn means that the particles have to travel longer distances (from one electrode to the other). Higher traveling distances means slower speed (slow response time) or increased electric field (high voltage) to accelerate particle migration from one plate to the other. Using both negatively and positively charged particles in the same microcapsule or cell to overcome this problem leads to aggregation (positively charged particles attracting, and sticking to, negatively charged particles).

It is known that the physical properties and surface characteristics of electrophoretic particles can be modified by adsorbing various materials to the surface of the particles, or chemically bonding various materials to the surface. U.S. Pat. No. 6,929,889 discloses pigment particles modified with surface organic groups to increase surface charge and stability in non-aqueous electrophoretic fluids, U.S. Pat. No. 4,891,245 describes a process for producing particles for use in EPDs, wherein solid pigment particles are coated with a polymeric material. U.S. Pat. No. 6,822,782 describes a process to produce polymer-coated particles for an electrophoretic medium in which the polymer is cross-linked around, or chemically bonded to the particle. U.S. Pat. No. 6,866,760 describes a process to produce droplets dispersed in an electrophoretic medium wherein a film-forming material forms a continuous phase surrounding and encapsulating the droplets. While all of these types of particles can be useful in EPDs with in-plane electrodes, they can involve complex process conditions, they suffer from light scatter due to a refractive index mismatch between the particle and the electrophoretic liquid, and they tend to settle due to a density-index mismatch between the particle and the electrophoretic liquid.

U.S. Pat. No. 5,582,700 describes display technology that uses liquid droplets (in a reverse emulsion) wherein polar liquid droplets containing a dye are dispersed in a transparent continuous non-polar liquid phase, wherein the distribution of the polar phase droplets dispersed in the non-polar phase is controlled electrophoretically. In the addressing method, the droplets are not moved from one plate to another but they are aggregated and dispersed within the continuous phase. It is possible that such an switching modality may have a more limited response times than desired.

Another issue with which to contend, in the case of particles dispersed in low density hydrocarbon solvents such as dodecane, is settling of the dispersed phase with time, as governed by Stoke's Law that defines settling velocities of particles in a fluid by the following equation:
V=[(2gr²)(d₁−d₂)]/9μ
where V=velocity of settling, g=acceleration due to gravity, r=radius of particle or dispersed phase, d₁=density of dispersed phase, d₂=density of medium, and μ=viscosity of the continuous phase. The issue of settling or creaming of particles is especially relevant to electro-optical modulating display devices in which particles are dispersed in a liquid system. It is important that the particles in such systems remain neutrally buoyant, neither creaming nor settling. Since viscosity and density mismatches of the dispersed phase, typically solid particles, and the continuous phase are usually so large, techniques such as increasing the viscosity of the continuous phase using polymeric additives are employed to overcome this effect. Such solutions, however, can result is potential drawbacks, for example, causing the electrical mobility of the particles to be compromised.

Therefore, there remains a need for electro-optical modulating display fluids that are improved in operation, where the particles are substantially neutrally buoyant, are non-scattering, and exhibit improved image quality, image stability, and resolution when used in an electro-optical modulating display device. A further need exists for a display system having an improved response rate, such as faster switching rates over EPDs using currently available particles and display fluids that can respond effectively to an electrical gate electrode.

### PROBLEM TO BE SOLVED BY THE INVENTION

The present invention intends to provide an electro-optical modulating display device comprising an array of pixels each containing at least one separate cell of electro-optical imaging fluid comprising an oil-in-oil (O/O) emulsion wherein one oil, dispersed in another immiscible oil, comprises a colorant, wherein the emulsion does not scatter light and provides a substantially common surface for all the colorants that are used in the display. It is also desired that the composition for the emulsion can be amenable to chemical modification, if necessary, and can be made by a simple process. It is a further object of the present invention to provide electro-optical modulating display systems employing electro-optical imaging fluids that are improved in operation, where the particles do not settle, are neutrally buoyant, and exhibit improved image quality, image stability, and resolution when used in an electro-optical modulating display device.

### SUMMARY OF THE INVENTION

The present invention is directed to overcoming one or more of the problems set forth above. Briefly summarized, according to one aspect of the present invention, an electro-optical modulating display device comprises an array of pixels, each containing at least one separate cell of electro-optical imaging fluid, wherein the electro-optical imaging fluid comprises a colloidally stable dispersion of an oil-in-oil emulsion containing a first oil phase dispersed as droplets in a continuous second oil phase, which droplets have a number median diameter of 10 nm to 5000 nm, wherein the first oil phase is substantially immiscible in the second oil phase. The first oil phase comprises a first oil composition that comprises one or more first oils and the second oil phase comprises a second oil composition comprising one or more second oils, wherein the first oil composition and the second oil composition are both non-polar liquids having a dielectric constant of less than 25. The first oil phase further comprises colorant (not excluding white or black colorant) and optionally a polymer, wherein the display is designed to operate with the dispersed first oil phase remaining in the dispersed phase.

The O/O (oil-in-oil) emulsions used as imaging fluids are colloidally stable, are substantially neutrally buoyant due to extremely low settling and creaming rates and preferably have a narrow particle size distribution. In one preferred embodiment, the two phases, the continuous and dispersed phases have matched refractive indices and the dispersed phase is colored differently than the continuous phase. Such O/O emulsions are advantageous for providing a substantially common surface for a variety of different colorants due to effective encapsulation of the colorants by the oil in the dispersed oil phase, thereby providing more predictable behavior across a given color series.

The term “oil” is defined as a liquid that is not miscible with water, preferably non-volatile, and soluble in ether.

The term “dielectric constant” refers to the measure of the ability of the material to support an electric field and is a measure of the polarity of the material. The dielectric constant ∈ of a medium is its ability to reduce the force of attraction F of charged particles q₁ and q₂ separated at distance r compared to vacuum. The dielectric constant 6 is defined here by the equation, F=q₁q₂/(∈×r). Dielectric constants for some familiar substances are: water, 80.4; methanol, 33.6; and benzene, 2.3. High dielectric constant solvents such as water usually have polar functional groups, and often, high dipole moments.

The term “phase” is meant to refer to the entire composition of the phase, including both liquid oil and any additives dissolved or dispersed therein. The terms “oil” or “fluid carrier” refer to the total organic solvent, or mixture of liquid organic solvents, included in an oil phase, which solvents are inherently liquid in pure form at room temperature, not including inherently solid materials dissolved or dispersed solids in the liquid. Depending on the context, various properties may refer to either the entire composition of a phase or only the oil in the phase.

### ADVANTAGEOUS EFFECT OF THE INVENTION

There are several advantages of using the O/O emulsions as the imaging fluid for electro-optical modulating displays. Firstly, the liquid particles or droplets in the emulsion remain substantially neutrally buoyant in the cell and have high mobility. Secondly, switching time is made faster since the particles are small, have high charge densities and exhibit no background conductivity. Thirdly, the emulsion shows consistent behavior in the cell with time, resulting in excellent aging behavior. Fourthly, the O/O emulsions provide an excellent gating window.

### DETAILED DESCRIPTION OF THE INVENTION

This invention relates to an electro-optical modulating display device comprising an array of individual pixel elements, each containing at least one separate cell of an electro-optical imaging fluid, wherein the electro-optical imaging fluid comprises liquid droplets, exhibiting a visibly contrasting color, dispersed in another liquid continuous phase, wherein the liquid droplets are immiscible with the continuous phase and capable of moving in an electric field. The liquid droplets preferably have the optimum particle size and density such that the particles are neutrally buoyant (do not settle or cream) in the continuous liquid medium, are colored with pigments or dyes, is refractive index matched with the continuous liquid, are non-scattering, and have a substantially common surface composition. The liquid droplets in the display medium may also optionally be modified with charge controlling agents to improve their electrically modulated mobility.

A pixel is defined herein as one or more spatially related and adjacent, independently controllable cells that contribute to the overall display structure. In such a pixel, the cells that make up the pixel may be in the plane of a single layer perpendicular to the direction of viewing or stacked upon each other in the direction of viewing. A cell is defined herein as the smallest structural unit of the electro-optical modulating display in which the movement of particles, which can result in the formation of color (or absence of a color) in the cell, is independently controlled relative to other elements of the display, wherein the cells are used in an array to form an image, which can be a digital image in which each pixel has two or more optical states, optionally including the control of density by partial migration of particles, enabled by the predictable mobility of the O/O emulsion, wherein at least one optical state is colored by the particles. Individual cells most commonly comprise a reservoir of imaging fluid and at least one pair of electrodes.

In one embodiment, the optical state of each cell can be controlled by the number density of particles in the viewing area of the cell. For example, dark particles may be assembled against a light (white) background, to display a desired image character upon control of the pixels and their associated cells.

Various designs for the cell, including various electrode designs, are known in the art, for example, cells with a top electrode and an opposing bottom electrode (referred to as an out-of-plane electrode arrangement). A display device with out-of-plane electrodes are also referred to as a “vertical migration type EPD device,” (see U.S. Pat. No. 6,741,385) or top/bottom electrode devices (US 2003/0035198). In a reflective EPD of this type, at least one of the electrodes is transparent and is always chosen to be the one facing, and closest to, a viewer. In an electro-optical modulating display utilizing out-of-plane electrodes, particles are typically plated at a top electrode to obtain a first optical (reflective) state or plated onto a bottom electrode to realize a second optical state. Accordingly, in the first optical state, the cell takes on the color of the charged particles that have plated onto the top electrode (which in this description is facing and closest to the viewer). In the second optical state, the cell takes on the color of the liquid containing a desired dye provided in sufficient optical density to absorb the light at the transparent top electrode. The particles assembled at the bottom electrodes are effectively hidden by the dye solution. However, in the first optical state, color contrast is reduced because the color is compromised by the presence of dye solution that remains on and between particles assembled at the top electrode.

In one embodiment, the cells are arranged as rows and columns. Row lines run along the rows of cells, and column lines run along the columns of cells. The row lines are connected to a row driver and the column lines to a column driver.

In a preferred embodiment, the individual pixel comprises a cell positioned between electrodes, wherein each cell is filled with an electro-optical imaging fluid. The cell can be of any suitable shape and can be made by any suitable process, including for example, partition walls vertically extending from a substrate or walls formed by encapsulation. Such cells can be made by various manufacturing techniques and are not limited in respect to their method of manufacture, which can include, among others, photolithographic, molding, or encapsulation techniques. In one embodiment, partition walls keeps the individual cells separate from one another. Any suitable wall design known in the art, or equivalents thereof, may be used. The width and/or diameter of the individual cells preferably have a largest diameter (in plan view) of from, for example, about 5 micrometers to about 1000 micrometers, preferably 10 to 200 micrometers. Obviously, the imaging fluid within the cells must contain particles of a size smaller than the pixel width/diameter in order to function. The solid portion of the wall separating the multiplicity or array of cells, i.e., the partitions between individual cells in an imaging layer, should preferably be as thin as possible. Preferred partition thicknesses are on the order of, for example, about 10 micrometers to about 100 micrometers, more preferably about 15 to about 50 micrometers (μm), although variations may exist depending on the particular display dimensions and use, for example, including displays for signs of various size.

The electro-optical modulating display device may have any suitable overall length and width as desired. The electro-optical modulating display device may also be made to have any desired height, although a total height of from about 5 to about 400 μm is preferred in terms of size and ease of use of the device.

Another type of EPD device involves an in-plane electrode arrangement in which, for a dark (colored) state, particles populate a viewing area between a collector electrode and a second in-plane electrode positioned to draw particles into the electrode-free viewing area. A voltage is only applied for a sufficient time to cause particles to fill in the viewing area, but not for duration that causes the particles to collect on the second electrode to a degree that de-populates particles from the viewing area. In a passive matrix drive scheme, intrinsic bistability, as described below, and threshold behavior allow such a scheme to work in a display device. In the absence of intrinsic bistable and threshold behavior of the particles, an electrical gate electrode may be used to prevent the unwanted migration of particles in non-selected cells. The term “threshold”, in the context of the present invention, is defined as the maximum bias voltage that may be applied to a cell without causing movement of particles between two electrodes on opposite sides of the cell. Threshold behavior is required to suppress or eliminate the undesirable crosstalk or cross-bias effect in adjacent cells of a passive matrix display. The possibility of using an electrical gate is desirable as it eliminates the difficult preparation of particle dispersions that are intrinsically bistable and intrinsically exhibit threshold behavior in combination with desired optical qualities and electrical robustness.

An EPD display device according to the present invention can have both in-plane and out-of plane electrodes or can be subject to modulating in addition to an electric field, for example, both an electrical and magnetic field. The EPD display device may comprise a stacked arrays of cells, particularly for colored displays. It will be understood by the skilled artisan that the present invention is not limited to any particular EPD design, but has general applicability to diverse embodiments, as well as to electrowetting and other types of displays in which an imaging fluid comprises dispersed liquid droplets that move in a second fluid during operation of the display.

This invention relates both to displays that are reflective or transmissive, based on electro-optical modulation of an imaging material derived from electrophoretic, electrochemical, electrochromic, electrowetting and/or liquid crystal effects. In a preferred embodiment, the electro-optical imaging material can be addressed with an electric field and then retain its image for a finite duration of sufficient length after the electric field is removed, a property typically referred to as “bistable”.

Particularly suitable electrically imagable materials that exhibit “bistability” are electrochemical, electrophoretic, electrochromic, magnetic, or chiral nematic liquid crystals. There are a number of different approaches, but they all share the ability to retain an image or position even when the power to the display has been turned off. This makes them especially useful for portable, battery-powered devices where the information on the display changes relatively infrequently.

As indicated above, the electro-optical modulating displays of the present invention comprises O/O compositions comprising droplets of a discontinuous oil phase containing a low dielectric, essentially non-volatile organic liquid, such as an organic phosphate liquid or a silicone oil, dispersed in a continuous phase of another low dielectric organic liquid such as an essentially non-volatile hydrocarbon. The composition for the discontinuous, or dispersed, oil phase further includes colorant. The dispersed phase in such emulsions typically have a number median diameter of less than about 5000 nm, have excellent stability to coalescence, and can be controlled to have a relatively very narrow particle size distribution.

The electro-optical imaging fluid used in the present invention is comprised of at least one set of colored oil droplets dispersed in at least one continuous fluid carrier. In some embodiments mixtures of differently colored liquid droplets or particles may be used, or liquid particles may be mixed with solid particles. The fluid carrier for the continuous oil phase can be chosen based upon properties such as dielectric constant, boiling point, and solubility, depending on the application. In one embodiment, a preferred fluid has a low dielectric constant (less than 10), a high boiling point (greater than 100° C. at atmospheric pressure) and viscosity less than 50 cP at 25° C. The discontinuous phase fluid preferably has a solubility in the continuous phase fluid of less than 1 percent by weight at room temperature. Further, to minimize the settling or creaming velocity of the dispersed phase in the O/O emulsion and maintain neutral buoyancy of the emulsion droplets, according to Stoke's Law, the difference in density between the discontinuous and continuous phases should be small and the number median particle size of the dispersed phase droplets should be sufficiently small.

The choice of oil for the continuous phase may further be based on chemical inertness and chemical compatibility with the dispersed oil phase. The viscosity of the fluid should be low when movement of the dispersed droplets is desired, such as when the emulsion is used in an electro-optical modulated field. For applications in which it is desired to optimize the light transmission through the O/O composition, it may be desired to minimize scattering by substantially matching the refractive index of the continuous phase fluid to that of the droplets. As used herein, the refractive index of the continuous phase or its carrier fluid “is substantially matched” to that of the dispersed phase or its carrier fluid if the difference between their respective refractive indices is between about zero and about 0.3, preferably between about 0.05 and about 0.2. Additionally, the fluid for the continuous phase may be chosen to be a poor solvent for some polymers or colorants which are incorporated into the dispersed oil phase, advantageously for the fabrication of droplets, because such a condition increases the range of materials that can be used in fabricating dispersions of droplets containing polymers and colorants.

Regarding the continuous phase, organic solvents, such as saturated linear or branched hydrocarbons of the general formula CₙH₂ₙ₊₂ where n can be between 6-20 or alkanes, aromatic hydrocarbons, halogenated organic solvents, and silicone oils are a few suitable types of liquid fluids for the continuous phase, which fluid may comprise a single fluid. The fluid, however, can also be a blend of more than one oils in order to tune its chemical and physical properties. Useful hydrocarbons include, but are not limited to, octane, decane, dodecane, tetradecane, xylene, toluene, naphthalene, hexane, cyclohexane, benzene, the aliphatic hydrocarbons in the ISOPAR series (Exxon), NORPAR (a series of normal paraffinic liquids from Exxon), SHELL-SOL (Shell), SOL-TROL (Shell), naphtha, and other petroleum solvents such as superior kerosene, paraffin oil, white mineral oil, molex raffinate, or suitable mixtures thereof. These materials usually have low densities. Useful examples of silicone oils include, but are not limited to, octamethyl cyclosiloxane and higher molecular weight cyclic siloxanes, poly(methyl phenyl siloxane), hexamethyldisiloxane, and polydimethylsiloxane. These materials also usually have low densities. Other useful organic solvents include, but are not limited to, epoxides, such as, for example, decane epoxide and dodecane epoxide; and vinyl ethers, such as, for example, cyclohexyl vinyl ether.

Furthermore, the continuous phase fluid may contain surface modifiers to modify the surface energy or charge of the dispersed oil droplets. Preferably, the fluid is clear or transparent and does not itself exhibit any color, although, again, such is not prohibited by the present invention as discussed above. The continuous phase is preferably a low-dielectric composition and substantially free of ions.

Oils for the dispersed or discontinuous phase in the O/O emulsions according to this invention are non-volatile, preferably non-polar liquids, preferably an organic phosphate liquid or a silicone oil in one embodiment. Preferred organic phosphate liquids includes, for example, branched or unbranched alkyl, cycloalkyl, alkylcycloalkyl, aryl, and alkylaryl phosphates-based solvents such as dialkyl, diaryl, trialkyl and triaryl phosphates, in which the organic groups may be substituted or unsubstituted, preferred substituents including non-polar groups such as halogens and ethers. In a preferred embodiment, each alkyl group of the di- or trialkyl phosphate has one to ten carbon atoms, more preferably two to eight carbon atoms. The aryl groups may be ring substituted such as, for example, in tricresyl phosphate. The alkyl or aryl groups of the di- or trialkyl and aryl phosphate can all be the same or can be different. A particularly preferred trialkyl phosphate is triethyl phosphate. Mixtures of different liquid organic phosphates, such as mixtures of dialkyl and trialkyl phosphates or diaryl and triaryl phosphates can be employed. Preferably, these phosphates have a boiling point greater than about 100° C. at atmospheric pressure, a dielectric constant less than 25, and a viscosity less than 100 cP at 25° C. and are substantially insoluble in the continuous phase. Further, after incorporation of polymers and optionally colorants in the dispersed oil phase liquids, it is preferred that the final viscosity be less than 200 cP and more preferably less than 100 cP at 25° C. for ease of dispersibility in the continuous phase.

The oil for the dispersed phase must be capable of being formed into small droplets in the continuous phase at the temperature at which the droplets are formed. Processes for forming small droplets include flow-through jets, membranes, nozzles, or orifices, as well as high shear emulsifiers and high-pressure homogenizers. The formation of small droplets may be assisted by the use of electrical or sonic fields.

One or more dispersants (including surfactants) can be used to aid in the stabilization and emulsification of droplets in the continuous phase. In one embodiment the dispersant is a compound (including polymers) that is soluble in the continuous phase and sparingly soluble in the dispersed phase and may be added to prevent particle flocculation. Dispersants useful in forming emulsions of the present invention include a variety of ionic and nonionic emulsifiers. In general, dispersants having multiple anchor sites to droplet walls have an advantage in effectively stabilizing the droplets. Blends of dispersants can be used to achieve the necessary requirements for emulsification and stabilization of the droplets and the necessary emulsion properties. In contrast to detergents, which have an HLB OF greater than 13, DISPERSANTS, OR WETTING AGENTS, are characterized by an HLB less than 10, preferably less than 7.

A partial listing representative of preferred dispersants for use in forming the O/O emulsions used in the displays of the invention includes poly(styrene-co-lauryl methacrylate-co-sulfoethyl methacrylate), poly(vinyltoluene-co-lauryl methacrylate-co-lithium methacrylate), poly(vinyltoluene-co-lauryl methacrylate-co-lithium methacrylate), poly(styrene-co-lauryl methacrylate-co-lithium methacrylate), poly(t-butylstyrene-co-styrene-co-lithium sulfoethyl methacrylate), poly(t-butylstyrene-co-lauryl methacrylate-co-lithium methacrylate), poly(t-butylstyrene-co-lithium methacrylate), poly(t-butylstyrene-co-lauryl methacrylate-co-lithium methacrylate-co-methacrylic acid), and poly(vinyltoluene-co-lauryl methacrylate-co-methacryloyloxyethyltrimethylammonium p-toluenesulfonate). Useful block or comb copolymers dispersants include, but are not limited to, AB diblock copolymers of (A) polymers of 2-(N,N-dimethylamino)ethyl methacrylate quaternized with methyl p-toluenesulfonate and (B) poly(2-ethylhexyl methacrylate), and comb graft copolymers with oil soluble tails of poly(12-hydroxystearic acid) and having a molecular weight of about 1800, pendant on an oil-soluble anchor group of poly(methyl methacrylate-methacrylic acid). Useful organic amides include, but are not limited to, polyisobutylene succinimides such as OLOA 11000, OLOA 1200 (Chevron), and N-vinylpyrrolidone polymers, including fatty acid salts of OLOA 11000 such as derived from oleic acid, myristic acid, stearic acid, and arachidic acid. Useful organic zwitterions include, but are not limited to, lecithin. Useful organic phosphates and phosphonates include, but are not limited to, the sodium salts of phosphated mono- and di-glycerides with saturated and unsaturated acid substituents. Examples of suitable polyester amine dispersants include SOLSPERSE 13940 (Noveon) and especially those described in GB-A-2001083, namely comprising the reaction product of a poly(lower alkylene)imine with a polyester having a free carboxylic acid group, in which there are at least two polyester chains attached to each poly(lower alkylene)imine chain. Mixtures of dispersants may be used if desired.

Particularly useful dispersants include compounds comprising at least two different segments, a first segment comprising heteroatoms for absorption to the dispersed phase and a second segment comprising continuous-phase soluble moieties. For example, the first segment may comprises amine groups for attachment and the second segment may comprise, for compatibility with the second phase, repeat units of a monomer. Such compounds are commercially sold under the trademarks OLOA 11000 and SOLSPERSE 13940 (polyesteramine(aziridine-hydroxy stearic acid copolymer), and poly(t-butylstyrene-co-lithium methacrylate). A preferred surfactant is OLOA 11000 a polyethyleneimine substituted succinimide derivative of polyisobutylene.

In another embodiment, instead of dispersants, solid particle stabilizers having a hydrophobic surface are used to aid in stabilization during or after emulsification of the dispersed phase in the continuous phase. The dispersed phase in such emulsions have a number median diameter of at least about 1 μM, have excellent stability to coalescence, and can be controlled to have a relatively very narrow particle size distribution. The emulsions can be formulated by a relatively simple, and inexpensive process.

Various inorganic particles, including metallic oxides such as alumina or silicon-containing oxides, surface treated with a hydrophobic material, may be suitably used. Alternately, suitable solid organic colloidal particles, for example, co-polymer particles such as described in U.S. Pat. No. 4,965,131 may be used as the solid particulate stabilizer.

A particularly preferred hydrophobically surfaced solid particle stabilizer is referred to as hydrophobic silica. Such silica particles have an average particle size of from 0.1 nm to 5 mm prior to homogenization with the oils. During homogenization, the silica particles break up and undergo a particle size reduction to less than 500 nanometers (nm), as measured by transmission electron microscopy. It is these particles that effectively surround and stabilize the disperse phase. The reduced hydrophobic silica particles have dimensions from about 10 to 300 nm and preferably from about 30 to 150 nm. The size and concentration of these particles control the size of the dispersed phase droplets. Although hydrophobic silicas are preferred, other hydrophobic or non-polar oil dispersible solid organic and/or inorganic particulates can be used, as mentioned above.

Hydrophobic silica for use in forming the O/O compositions of this invention include various fumed silicas that have been surface treated with reactive silicon-containing compounds such as commercially available silating agents that can impart hydrophobicity to the silica surface. Particularly useful hydrophobic silicas include NANOGEL and CAB-O-SIL TS 610 from Cabot Corporation. Blends of silicas can also be used to achieve the necessary stabilization.

Suitably, the hydrophobically surfaced solid particles are present at a concentration of from 5 to 75 weight percent with respect to the dispersed oil phase, preferably in an amount of from 5 to 50 weight percent of the dispersed oil phase.

In this embodiment, the hydrophobically surfaced solid particle stabilizer is preferably used in conjunction with a co-stabilizer that is soluble in the continuous oil phase. More specifically, the co-stabilizer promotes or enhances the adsorption of the hydrophobically surfaced solid particle stabilizer at the interface of the disperse phase oil droplets and the non-polar continuous oil phase. In particular, this combination of co-stabilizer and particle stabilizer, aids in keeping the dispersed phase droplets well dispersed in the continuous phase, thereby prolonging the shelf life of the O/O composition, especially when containing a dispersion of the one non-polar oil in another. Any suitable co-stabilizer that is soluble in the continuous organic phase and favorably affects the surface energetics of the solid particle stabilizer in the continuous phase may be employed in order to drive the solid particle stabilizer to the interface between the dispersed phase liquid droplets and the continuous phase. Such compounds can comprise at least two different segments or moieties, a first segment comprising moieties attracted to the dispersed phase and a second segment comprising continuous-phase soluble moieties. For example, a first segment may comprise amine groups and a second segment may comprise repeat units of an non-polar monomer, for example, isobutylene or the like. Useful co-stabilizers include for example, those compounds commercially sold under the trademarks OLOA (Chevron) and Solsperse (Noveon). Solsperse 13940, for example, is a polyesteramine(aziridine-hydroxy stearic acid copolymer. A preferred co-stabilizer is OLOA 11000 which is a polyethyleneimine substituted succinimide derivative of polyisobutylene.

Still another class of co-stabilizers is derived from small organic amine containing molecules, particularly, heterocyclic amines. Some preferred examples are, N-(1-acetyl-2,2,6,6-tetramethyl-4-piperidinyl)-2-dodecylsuccinimide (SANDUVOR 3058); 2-dodecyl-N-(2,2,6,6-tetramethyl-4-piperidinyl)-succinimide (SANDUVOR 3055); and 2-dodecyl-N-(1,2,2,6,6-pentamethyl-4-piperidinyl)-succinimide (SANDUVOR 3056).

Generally, the co-stabilizer is used in an amount of from 1 to 15 percent by weight of the solid particle stabilizer and more preferably from 1-10 percent by weight.

The imaging fluid system may be colored by any suitable means in the art, including through the inclusion of any suitable colorants (e.g., dyes and/or dispersible pigments) therein.

The dispersed phase of the O/O composition can, and preferably does, include useful ingredients including at least one colorant, for example, a pigment, a polymer, a laked pigment, a dye, a pigment-polymer composite, a dye-polymer composite or some combination of the above. Preferably the pigment, polymer, and/or pigment-polymer composite is present in the dispersed oil phase in a total amount of from 1 to about 50 percent by weight of the dispersed phase, and oil in the dispersed phase is present in the amount of from 50 to 99 percent by weight of the dispersed phase. In one embodiment, the dispersed oil phase comprises colorant (including pigment or dye) in an amount 1 to 30 percent, preferably 1 to 15 percent, by weight of the dispersed first oil phase and 0.1 to 60 percent, preferably 1 to 40 percent, by weight of one or more polymers molecularly dissolved in the dispersed oil phase. A pigment, laked pigment, or pigment-polymer composite, in order to be dispersed in the dispersed phase, should have an average particle diameter sufficiently small relative to the diameter of the dispersed first oil phase, preferably an average particle diameter on average 10 to 100 nm.

In one embodiment, a pigment-polymer composite may be formed by a physical process such as melt-compounding the polymer and colorant, followed by grinding, attrition, or ball milling. Such composites have been previously used for making conventional xerographic toners and are well known in the art, including the polymers and colorants used to make such toners, and are commercially available from any number of suppliers. A pigment-polymer composite can be mixed into the oil fluid for the dispersed phase by stirring in the composite until the polymer dissolves in the oil. The pigment may also be milled in the oil for the dispersed phase with or without the polymer present. The pigment in the pigment-polymer composite may be present in an amount of from 0.1 to 80 percent by weight of the pigment-polymer composite. The pigment-polymer composite can be used in amounts of from 1 to about 50 percent by weight of the dispersed phase, preferably from 5-30 percent by weight, and most preferably from 10-25 percent by weight.

Polymers useful for incorporation in the oil droplets preferably are oil-soluble resins and include, but are not limited to, homopolymers and copolymers such as polyesters, styrenes, e.g. styrene and chlorostyrene; monoolefins, e.g. ethylene, propylene, butylene and isoprene; vinyl esters, e.g. vinyl acetate, vinyl propionate, vinyl benzoate and vinyl butyrate; α-methylene aliphatic monocarboxylic acid esters, e.g. methyl acrylate, ethyl acrylate, butyl acrylate, dodecyl acrylate, octyl acrylate, phenyl acrylate, methyl methacrylate, ethyl methacrylate, butyl methacrylate and dodecyl methacrylate; vinyl ethers, e.g. vinyl methyl ether, vinyl ethyl ether and vinyl butyl ether; and vinyl ketones, e.g. vinyl methyl ketone, vinyl hexyl ketone and vinyl isopropenyl ketone and mixtures thereof. Particularly desirable binder resins include polystyrene resin, polyester resin, styrene/alkyl acrylate copolymers, styrene/alkyl methacrylate copolymers, styrene/acrylonitrile copolymer, styrene/butadiene copolymer, styrene/maleic anhydride copolymer, polyacrylonitrile resin, polyethylene resin and polypropylene resin and mixtures thereof. They further include polyurethane resin, epoxy resin, silicone resin, polyamide resin, polycaprolactone resin, modified rosin, paraffins and waxes and mixtures thereof. In a preferred embodiment the resins most preferred for the O/O compositions are polyesters and are soluble in the oil for dispersed phase. Suitable polyester resins include polyesters derived from bisphenol A. One preferred polymer is a polyester, for example, TUFTONE NE-303 (Kao Corporation), a polyester copolymer of bis-phenol A.

Optional polymers for the dispersed phase may be selected based on the desired properties to be imparted by the inclusion of the polymers, depending on the particular application. For example, a polymer may be used that is designed or preselected to be functionalized with a charged group in order to control mobility of the dispersed phase through the continuous phase when the emulsion composition is subjected to an electric or magnetic field. The polymer may also be selected to affect the viscosity of the dispersed oil-phase droplets.

Dyes for use in the dispersed droplets in the imaging fluid can be a pure compound, or blends of dyes to achieve a particular color, including black. The dyes can be fluorescent. photoactive, changing to another color or becoming colorless upon irradiation with either visible or ultraviolet light. Dyes could also be polymerizable by, for example, thermal, photochemical or chemical diffusion processes, forming a solid absorbing polymer inside the droplet. Properties desired for the dyes include light fastness, solubility in the suspending liquid, and color. Low cost is a factor. These dyes are generally chosen from the classes of azo, anthraquinone, and triphenylmethane type dyes and may be chemically modified so as to increase their solubility in the oil phase. Useful azo dyes include, but are not limited to: the Oil Red dyes and the SUDAN Red and SUDAN Black series of dyes. Useful anthraquinone dyes include, but are not limited to: the Oil Blue dyes, and the MACROLEX Blue series of dyes. Useful triphenylmethane dyes include, but are not limited to, Michler's hydrol, Malachite Green, Crystal Violet, and Auramine O.

A neat pigment can be any pigment, and, usually for a light colored particle, pigments such as rutile (titania), anatase (titania), barium sulfate, kaolin, or zinc oxide are useful. Some typical particles have high refractive indices, high scattering coefficients, and low absorption coefficients. Other particles are absorptive, such as carbon black or colored pigments used in paints and inks. The pigment should also be insoluble in the continuous phase. Yellow pigments such as diarylide yellow, HANSA yellow (Clariant), and benzidine yellow have also found use in similar displays. Any other reflective material can be employed for a light colored particle, including non-pigment materials, such as metallic particles.

Useful neat pigments include, but are not limited to, PbCrO4, SUNFAST Blue 15:3, SUNFAST Magenta 122, Cyan blue GT 55-3295 (American Cyanamid Company, Wayne, N.J.), CIBACRON Black BG (Ciba Company, Inc., Newport, Del.), CIBACRON Turquoise Blue G (Ciba), CIBALON Black BGL (Ciba), ORASOL Black BRG (Ciba), ORASOL Black RBL (Ciba), Acetamine Black, CBS (E.I. DuPont de Nemours and Company, Inc., Wilmington, Del., hereinafter abbreviated “DuPont”), CROCEIN SCARLET N EX (DuPont) (27290), Fiber Black VF (DuPont) (30235), LUXOL Fast Black L (DuPont) (Solv. Black 17), NIROSINE Base No. 424 (DuPont) (50415 B), Oil Black BG (DuPont) (Solv. Black 16), Rotalin Black RM (DuPont), SEVRON Brilliant Red 3B (DuPont); Basic Black DSC (Dye Specialties, Inc.), HECTOLENE Black (Dye Specialties, Inc.), AZOSOL Brilliant Blue B (GAF, Dyestuff and Chemical Division, Wayne, N.J.) (Solv. Blue 9), AZOSOL Brilliant Green BA (GAF) (Solv. Green 2), AZOSOL Fast Brilliant Red B (GAF), AZOSOL Fast Orange RA Conc. (GAF) (Solv. Orange 20), AZOSOL Fast Yellow GRA Conc. (GAF) (13900 A), Basic Black KMPA (GAF), BENZOFIX Black CW-CF (GAF) (35435), CELLITAZOL BNFV EX Soluble CF (GAF) (Disp. Black 9), CELLITON Fast Blue AF EX Conc (GAF) (Disp. Blue 9), CYPER Black IA (GAF) (Basic Black 3), DIAMINE Black CAP EX Conc (GAF) (30235), DIAMOND Black EAN Hi Con. CF (GAF) (15710), DIAMOND Black PBBA EX (GAF) (16505); DIREC Deep Black EA Ex CF (GAF) (30235), HANSA Yellow G (GAF) (11680); INDANTHRENE Black BBK Powd. (GAF) (59850), INDOCARBON CLGS Conc. CF (GAF) (53295), KATIGEN Deep Black NND Hi Conc. CF (GAF) (15711), RAPIDOGEN Black 3 G (GAF) (Azoic Black 4); SULPHONE Cyanine Black BA-CF (GAF) (26370), ZAMBEZI Black VD Ex Conc. (GAF) (30015); RUBANOX Red CP-1495 (The Sherwin-Williams Company, Cleveland, Ohio) (15630); REGAL 330 (Cabot Corporation), RAVEN 11 (Columbian Carbon Company, Atlanta, Ga.), (carbon black aggregates with a particle size of about 25 μm), STATEX B-12 (Columbian Carbon Co.) (a furnace black of 33 μm average particle size), and chrome green.

Laked pigments are particles that have a dye precipitated on them and are metal salts of readily soluble anionic dyes. These are dyes of azo, triphenylmethane or anthraquinone structure containing one or more sulphonic or carboxylic acid groupings. They are usually precipitated by a calcium, barium or aluminum salt onto a substrate. Typical examples are PEACOCK BLUE lake (Cl Pigment Blue 24) and PERSIAN ORANGE (lake of Cl Acid Orange 7), BLACK M TONER (GAF) (a mixture of carbon black and black dye precipitated on a lake).

The pigment-polymer composite may also contain, in addition to the pigment and polymer, other additives such as organo-cations, for example, quaternary ammonium and phosphonium compounds. Specific examples of these include, but are not limited to, lauramidopropyltrimethylammonium methylsulfate, octadecyldimethylbenzylammonium m-nitrobenzenesulfonate, methyltriphenylphosphonium tetrafluoroborate, and methyltriphenylphosphonium tosylate.

In one embodiment, the process for making the O/O emulsion is carried out, for example, by combining a pigment-polymer composite dispersed in the oil for the discontinuous phase with the oil for the continuous phase, such that the discontinuous phase is present at a weight percent of 1-50 weight percent, preferably 5-40 weight percent of the continuous phase and mixing the ingredients using shear force, for example a homogenizer at room temperature until an O/O emulsion is formed. Any type of mixing and shearing equipment may be used to perform these steps such as a batch mixer, planetary mixer, single or multiple screw extruder, dynamic or static mixer, colloid mill, high pressure homogenizer, sonicator, or a combination thereof. While any high shear type agitation device is applicable to the process of this invention, a preferred homogenizing device is the MICROFLUIDIZER such as Model No. 110T produced by Microfluidics Manufacturing. In this device, the droplets of the first oil phase (discontinuous phase) are dispersed and reduced in size in the second oil phase (continuous phase) in a high shear agitation zone and, upon exiting this zone, the particle size of the dispersed oil is reduced to uniform sized dispersed droplets in the continuous phase. The temperature of the process can be modified to achieve the optimum viscosity for emulsification of the droplets. The number median particle size of the O/O emulsion droplets is not more than 5000 nm and preferably less than 3000 nm but at least 10 nm, more preferably at least 25 nm.

In one embodiment of the invention, the electro-optical modulating display fluid comprises one set of colored oil droplets dispersed in a colored continuous phase, the droplets exhibiting different, contrasting color to the color of the continuous phase.

The invention will further be illustrated by the following examples:

### EXAMPLES

TUFTONE NE-303, a bisphenol A polyester resin polymer, used in the examples below was obtained from Kao Specialties Americas LLC a part of Kao Corporation, Japan. The carbon black pigment REGAL 330 used in the examples was obtained from Cabot Corporation, Billerica, Mass. SUNFAST Blue 15:3 (PB 15:3) was obtained from Sun Chemicals. Triethyl phosphate (TEP) and n-dodecane were purchased from Aldrich Chemical Co., Milwaukee, Wis. OLOA 11,000 a polyisobutylene succinimide, 62% active in mineral oil, was obtained from Chevron in San Ramon, Calif.

### Example 1

Preparation of the Pigmented O/O Emulsions:

A pigment-polymer composite (4 g) comprising 25 weight % REGAL 330 and 75 weight % TUFTONE NE-303 polymer was dissolved in 16 grams of TEP at ambient temperature. This was dispersed in 76 g of dodecane containing 4 g of OLOA 11000 (100% active) such that the ratio of the dispersed phase to the dispersant is 5:1 using an overhead SILVERSON L4R mixer from Silverson for one minute at maximum speed. The resultant dispersion was homogenized using a MICROFLUIDIZER Model #11 OT from Microfluidics at a pressure of 12,000 lbs/sq inch until a fine dispersion was obtained.

The number median D(n), particle size was measured using a MALVERN Zetasizer ZS that uses low angle laser light scattering method and a 633 nm wavelength, 4 mW He—Ne laser. D(n) is the particle size which divides the population exactly into two equal halves such that there is 50% distribution above this value and 50% below. The number median particle size of the final O/O emulsion was determined to be 266 nm.

### Example 2

The same method as for Example 1 was used to make the O/O dispersion of Example 2 containing PB 15:3 except that REGAL 330 was replaced with the pigment-polymer composite of PB 15:3. The number median particle size of the final O/O emulsion from Example 2 was determined to be 233 nm.

Both the O/O emulsions. Examples 1 and 2, were observed to be negatively charged and the zeta potentials measurements in dodecane at 40V gave values of −41 mV and −34 mV for Examples 1 and 2, respectively as measured using the MALVERN Zetasizer ZS

### Comparative Example 3

Preparation of Milled Carbon Black

A dispersion of REGAL 330 in Isopar L was prepared by combining 2.5 g REGAL 330 carbon black, 12.5 g of a 20 wt % active OLOA 11000 solution in dodecane, 10.0 g dodecane (Fluka Corp), and 60 mL 1.8 mm zirconium oxide beads in a 4 oz glass jar. The jar was rolled for 5 days at a speed of 21 m/min to mill down the carbon black. After milling, the dispersion was separated from the beads. The median particle size of the final dispersion was determined to be 120 nm by light scattering, and microscopic examination of the dispersion showed all particles to be well dispersed. These particles were also observed to be negatively charged and a zeta potential value in dodecane of −37 mV was obtained at 40 V.

Performance Characterization of the O/O Emulsions in Electro-Optically Modulated Test Cells:

1. Particle Mobility Test:

The performance of the O/O emulsions from Examples 1 and 2 in electro-optically modulated test cells were studied by filling 180 μm square by 10 μm deep test cells having a lower planar glass surface and two 40 μm wide parallel indium tin oxide (ITO) electrodes, separated by 100 μm, with the each emulsion, covering with a second glass surface and sealing. The ITO electrodes were connected to a variable voltage source. The test cell was illuminated in transmission and subjected to a series of voltages and frequencies.

The response of the emulsion particles was video recorded with a frame grabber via the microscope. An electric field was applied to collect the particles at one side of the cell, and then the field was reversed to move the particles across the cell to the other electrode.

The percent optical transmission in a specific area of the 100 μm gap and the transition time were determined using image processing. Optical transmission measurements of greater than 90% before and after switching showed a high clearing efficiency.

The particle mobility was relatively constant over the range of 4 V to 40 V. The O/O emulsion was not prone to formation of vortices or turbulent flows in the video recording, even at high voltages up to 40 V, resulting in reduced switching times. This same behavior was exhibited by the O/O emulsion of Example 2. Examples 1 and 2 demonstrated low background conductivity of the continuous phase, indicating again the ability to drive the cell containing the O/O emulsions at high voltages without initiating field driven fluid flows (turbulence). These emulsions also had high charge densities. The comparative Example 3 on the other hand was slow, had low charge densities and required a very high voltage for particle movement at which point there was severe turbulence in the cell, making measurements impossible.

2. Gating Test:

This test was performed in a cell similar to the one described above except that the cell had 5 parallel, 10 um wide, electrodes. The 4^{th} electrode was grounded and the 2^{nd} and 3^{rd} were connected to variable voltage sources with a common ground. The particles were migrated to a location in the cell beyond the (3^{rd}) gating electrode, after which the voltage of the 3^{rd} electrode was elevated. To test for the gating window, voltage was gradually applied to the (2^{nd}) collector electrode until the particles were successfully pulled across the gate. The gating window was thus determined. The particles demonstrated a 19 V gating window when the 3^{rd} electrode was at 40 V.

3. Aging Test

An aging test to determine the stability of the O/O emulsion used in a cell was conducted in a 2-electrode cell as used for the mobility test experiment using the O/O emulsion from Example 2 over a 21 day interval. The test cell was filled with the O/O emulsion of Example 2 such that a 0.3 transmission density was obtained.

Information extracted from the 2-electrode cell tests included initial and final percent transmission, minimum transmission (peak optical density), and time to reach minimum transmission. In all instances, the initial and final transmission values were greater than 90%, enabling the 90%-90% clearing time metric to be determined.

Qualitatively, these tests showed a high initial transmission for the pre-cleared pixel, followed by a rapid decrease in transmission to a minimum value as the gap area was filled with particles, followed by a gradual increase in transmission to the original value as particles collected on the opposite electrode.

Particle transition time was considered to be the time period where the percent transmission was below a threshold level of 90% transmission. Particle speed was determined by the reciprocal of the transition time T. When particle speed was plotted against test (drive) voltage, the slope of the response was considered an indicator of particle mobility. Average mobility of 3.7×10⁻³ and 2.0×10⁻³ V⁻¹s⁻¹ for the 2-day and 21-day tests respectively were obtained for the sample from Example 2 over the 4-40 V drive voltage range, indicating no degradation of the emulsion in the cell with time. Table 1 shows the 2 and 21-day cell behavior including particle speed.

TABLE 1

| | | |
|---|---|---|
| Drive | Initial % Transmission | 1/T (sec⁻¹) |

| Voltage (V) | 2 day | 21 day | 2 day | 21 day |
|---|---|---|---|---|
| 4 | 44 | 48 | 0.01 | 0.01 |
| 10 | 11 | 18 | 0.03 | 0.02 |
| 20 | 11 | 11 | 0.06 | 0.04 |
| 40 | 8 | 7 | 0.14 | 0.08 |

The results described in Table 1 show that the mobility of the particles in the O/O emulsion did not deteriorate in the cell, as indicated by consistent performance over 21 days of repeated testing. The negatively charged particles showed no tendency for trapping at the positive electrode. The sign of particle charge was consistent over the 21-day time period and the mobility and clearing in the dense central region of the cell remained essentially the same as for the 2-day test. The emulsion after 21 days was still negative and highly mobile. Clearing level had not degraded over time and remained excellent. Comparison of videos captured during transport showed that the flow patterns and boundary conditions did not change. Aging influence was minimal compared to carbon black suspensions.

Aging studies performed using samples such as from Comparative Example 3 showed aging effects where the dispersion turns “sluggish” with time in the filled cell. This led to poor clearing and longer transition times. The time domain for a practically significant degradation for these dispersions was on the order of 1-2 weeks.

The invention has been described with reference to a preferred embodiment. However, it will be appreciated that variations and modifications can be effected by a person of ordinary skill in the art without departing from the scope of the invention.

1. An electro-optical modulating display device comprising an array of pixels, each pixel associated with at least one cell of electro-optical imaging fluid wherein the electro-optical imaging fluid comprises a colloidally stable oil-in-oil emulsion containing a first oil phase dispersed as droplets in a continuous second oil phase, which droplets have a number median diameter of 10 nm to 5000 nm, wherein the first oil phase is substantially immiscible in the second oil phase, the first oil phase comprising a first oil composition that comprises one or more first oils and the second oil phase comprising a second oil composition comprising one or more second oils, wherein the first oil composition and the second oil composition are both liquids having a dielectric constant of less than 25, the first oil phase further comprising colorant, not excluding black or white, and optional polymer, wherein the display is designed to operate with the dispersed first oil phase remaining in the dispersed phase during operation of the display.

2. The electro-optical modulating display device of claim 1 wherein each cell is partitioned from adjacent cells by partition walls.

3. The electro-optical modulating display device of claim 1 wherein each cell is formed by encapsulation in a non-fluid matrix.

4. The electro-optical modulating display device of claim 1 wherein each pixel corresponds to a single cell comprising at least one pair of electrodes capable of producing an electric field that changes the optical state of the electro-optical modulating imaging fluid in the cell.

5. The electro-optical modulating display device of claim 1 wherein each dispersed oil phase comprises a pigment or dye colorant.

6. The electro-optical modulating display device of claim 5 wherein each dispersed oil phase comprises a pigment colorant.

7. The electro-optical modulating display device of claim 1 wherein each dispersed oil phase comprises at least one polymer.

8. The electro-optical modulating display device of claim 7 wherein each dispersed oil phase comprises a pigment-polymer composite.

9. The electro-optical modulating display device of claim 1 wherein the dispersed first oil phase droplets are stabilized by an effective amount of dispersant.

10. The electro-optical modulating display device of claim 1 wherein the dispersed first oil phase droplets are stabilized by substantially covering the droplets with hydrophobically surfaced solid particulates.

11. The electro-optical modulating display device of claim 1 wherein refractive index of the continuous second oil phase is substantially matched to that of the dispersed first oil phase such that the difference between the respective refractive indices of the phases is between about zero and about 0.3.

12. The electro-optical modulating display device of claim 1 wherein the dielectric constant of the first and the second oils, respectively, in the two phases are both independently less than 25, before the addition of any solid additives to the oil compositions of the phases.

13. The electro-optical modulating display device of claim 1 wherein the continuous second oil phase has a dielectric constant less than 10.

14. The electro-optical modulating display device of claim 1 wherein the second oil phase comprises one or more solvents selected from the group consisting of substantially non-polar hydrocarbons, substituted or unsubstituted, including C₆-C₂₀ alkanes, substituted or unsubstituted aromatic hydrocarbons, and mixtures thereof.

15. The electro-optical modulating display device of claim 1 wherein the first oil composition comprises at least one liquid organic phosphate compound.

16. The electro-optical modulating display device of claim 15 wherein the liquid organic phosphate compound has a boiling point greater than about 100° C. at atmospheric pressure, a dielectric constant less than 25, and a viscosity less than 100 cP at 25° C.

17. The electro-optical modulating display device of claim 1 wherein the first oil phase, including the first oil composition and optional polymers, colorants or other additives, has a viscosity less than 200 cP at 25° C.

18. The electro-optical modulating display device of claim 1 wherein the dispersed first oil phase comprises colorant in an amount 1 to 30 percent by weight of the dispersed first oil phase and wherein the dispersed first oil phase is present in the amount of 1 to 50 weight percent of the continuous second oil phase.

19. The electro-optical modulating display device of claim 1 wherein the display is either a transmissive or reflective display.

20. A method of forming an image by movement of liquid droplets through a continuous liquid phase, the method comprising:
(a) providing an electro-optical modulating display device comprising an array of pixels, each pixel associated with at least one cell of electro-optical imaging fluid comprising a colloidally stable dispersion of an oil-in-oil emulsion containing a first oil phase dispersed as droplets in a continuous second oil phase, which droplets have a number median diameter of 10 nm to 5000 nm, wherein the first oil phase is substantially immiscible in the second oil phase, the first oil phase comprising a first oil composition that comprises one ore more first oils and the second oil phase comprising a second oil composition comprising one or more second oils, wherein the first oil composition and the second oil composition are both liquids having a dielectric constant of less than 25, the first oil phase further comprising colorant and optionally polymer, and
(b) applying an electric field to the oil-in-oil emulsion to cause the first oil phase droplets dispersed in the continuous second oil phase to move in either a first or second direction, depending on the polarity of the electrical field, between at least one pair of electrodes, such that the electro-optical imaging fluid changes its visible color, not excluding black or white color, wherein the dispersed first oil phase remains in the dispersed phase during operation of the display.
